(19) United States
(12) Patent Application Publication
(10) Pub. No.: US 2024/0226212 A1
(43) Pub. Date: Jul. 11, 2024

(54) GENOMIC SEQUENCES FOR YEAST PROBIOTICS

(71) Applicant: Worcester Polytechnic Institute, Worcester, MA (US)

(72) Inventors: Reeta Rao, Newton, MA (US); Eric M. Young, Worcester, MA (US); Lohith Kunyeit, Worcester, MA (US); K. A. Anu-Appaiah, Karnataka (IN); Joseph H. Collins, Worcester, MA (US)

(21) Appl. No.: 18/404,140

(22) Filed: Jan. 4, 2024

Related U.S. Application Data
(60) Provisional application No. 63/437,240, filed on Jan. 5, 2023.

(51) Int. Cl. A61K 36/064; A01N 63/32; A01P 3/00; A61P 31/10

(52) U.S. Cl. CPC A61K 36/064; A01N 63/32; A01P 3/00; A61P 31/10

(57) ABSTRACT

A formulated composition for treating or preventing epithelial fungal infection and correcting dysbiosis in or on a subject provides an effective dose of yeast cells containing a plurality of genes encoding functions of tolerance to a high temperature, tolerance to high and low pH, adherence to cells of the subject, and biosynthesis of amino acid alcohols.

### RELATED APPLICATIONS

This patent application claims the benefit under 35 U.S.C. § 119(e) of U.S. Provisional Patent App. No. 63/437,240, filed Jan. 5, 2023, entitled “GENOMIC SEQUENCES FOR YEAST PROBIOTICS,” incorporated herein by reference in entirety.

### STATEMENT OF FEDERALLY SPONSORED RESEARCH AND DEVELOPMENT:

This patent application was developed, either in whole or in part, with U.S. Government support under Contract No. 1R15AT009926-01, awarded by the National Institute for Health (NIH). The Government has certain rights in the Invention.

### STATEMENT OF COLOR DRAWINGS

The patent or application contains at least one drawing executed in color. Copies of this patent or patent application publication which color drawings(s) will be provided by the Office upon request and payment of necessary fee.

### BACKGROUND

Probiotic yeasts uniquely combine human health benefits and tolerance phenotypes that enable survival in the gastrointestinal tract. Perhaps unsurprisingly, probiotic yeasts are frequently found in fermented foods and beverages. Production conditions and consumption of fermented products promote the acquisition of desired phenotypes—isolates from fermented foods have been shown to tolerate high temperature, low pH, bile, and gastric and pancreatic enzymes, and have the ability to adhere to gut epithelial cells. Furthermore, probiotic yeasts can produce beneficial metabolites and inhibit bacterial and fungal infections.

### SUMMARY

A formulated composition for treating or preventing epithelial fungal infection and correcting dysbiosis in or on a subject provides an effective dose of yeast cells containing a plurality of genes encoding functions of tolerance to a high temperature, tolerance to high and low pH, adherence to cells of the subject, and biosynthesis of amino acid alcohols.

Probiotic yeast are emerging as preventative and therapeutic solutions for disease. Found in fermented beverages around the world, they survive harsh conditions of the gastrointestinal tract and inhibit pathogens like Candida albicans. Yet, little is known of the genetic determinants of these phenotypes. To this end, configurations herein nanopore sequence two candidiasis-mitigating probiotic yeast isolates—Saccharoymyces cerevisiae KTP and Issatchenkia occidentalis ApC. Phylogenetic analysis places strain KTP in a small clade that lacks any apparent ancestry from common European/wine Saccharomyces cerevisiae strains. Further analysis reveals that KTP genes involved in stress, acid tolerance, and adherence are markedly different from the type strain S. cerevisiae S288C—rather they resemble the probiotic yeast species Saccharomyces boulardii. Finally, we confirmed that common yeast genetic parts function in strain KTP. This work advances the genomics of Issatchenkia yeasts and establishes a strong genetic link among probiotic Saccharomycetes.

The same features that proliferate in the gut also provide efficacy for treatment of yeast infections, particularly in the human vaginal tract. It is estimated that 75% of women will experience an episode of Candida vaginitis once in their lifetime and approximately 3,871 cases per 100,000 women will suffer from recurrent vulvovaginal candidiasis. Current antifungal drugs show limited efficacy against acute disease and long-term usage of antifungals tend to have debilitating side effects. There is an unmet need to augment therapeutics options for vaginal candidiasis.

Recurrent vulvovaginal candidiasis often exhibits long-lasting symptoms requiring sustained regimen of antifungal therapy that drives the evolution of drug-resistant “super bugs” that are a clear and present danger to public health that has even been capitalized on by media and entertainment industries.. It is estimated that the US spends $55 billion each year to manage complications associated with—antimicrobial resistance.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

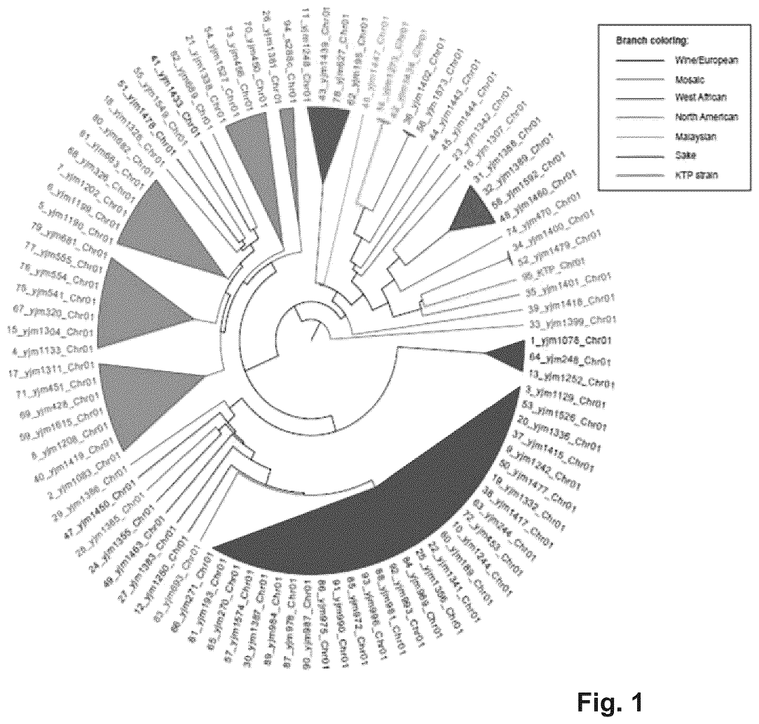

FIG. 1 is a global phylogenetic position of the KTP strain in comparison to other S. cerevisiae strains;

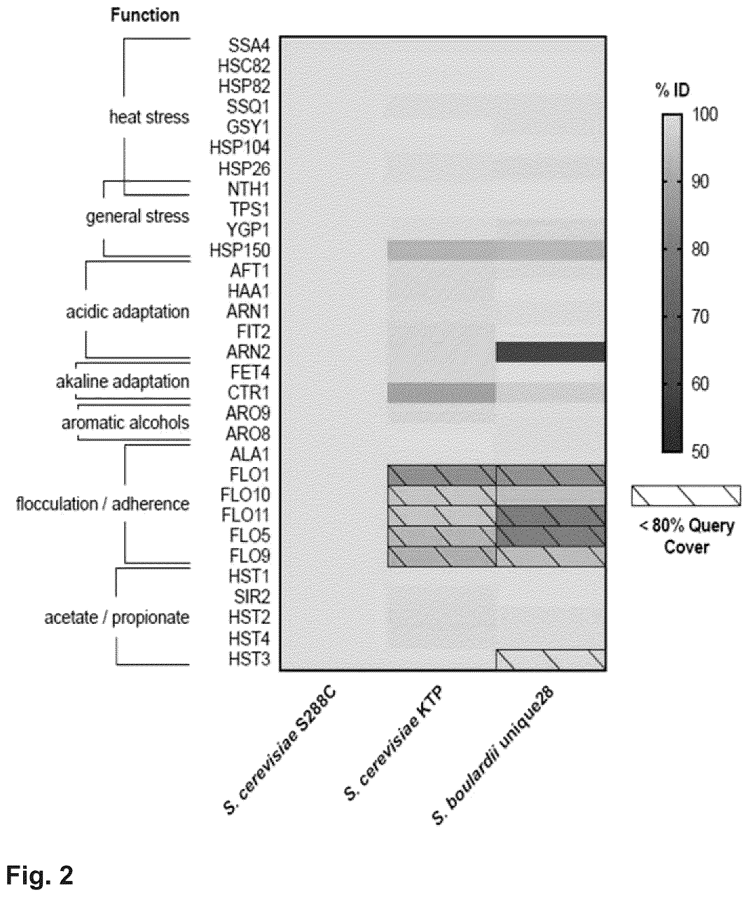

FIG. 2 shows a percent identity heatmap of BLASTp hits to known S. cerevisiae S288C probiotic-related proteins.

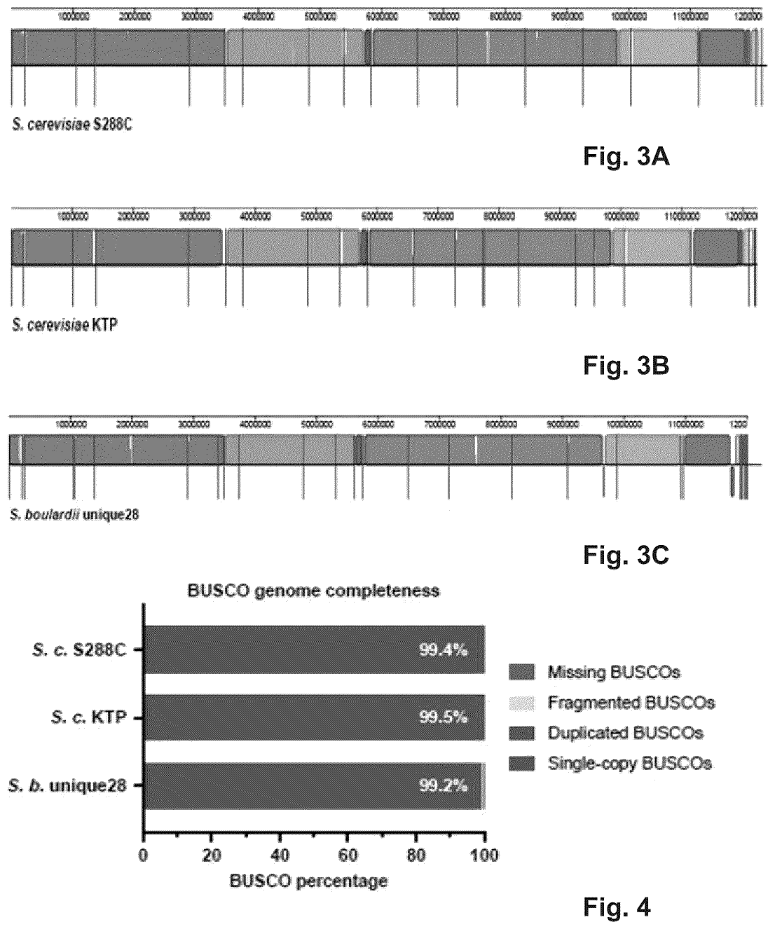

FIGS. 3A-3C show comparative genomics between the three strains S. cerevisiae S288c, S. cerevisiae KTP, and S. boulardii unique28 for whole genome alignment with ProgressiveMauve;

FIG. 4 shows BUSCO genome completeness assessment of the three strains of FIGS. 3A-3C;

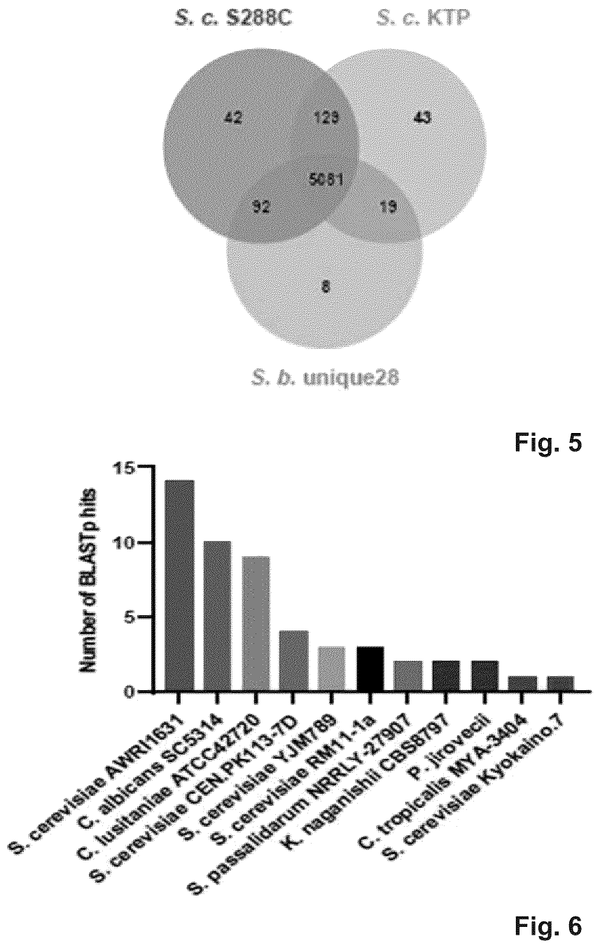

FIG. 5 shows a Venn diagram of orthologous proteins shared or unique to the three strains;

FIG. 6 shows the number of BLASTp hits for the unique KTP proteins according to the top strain found;

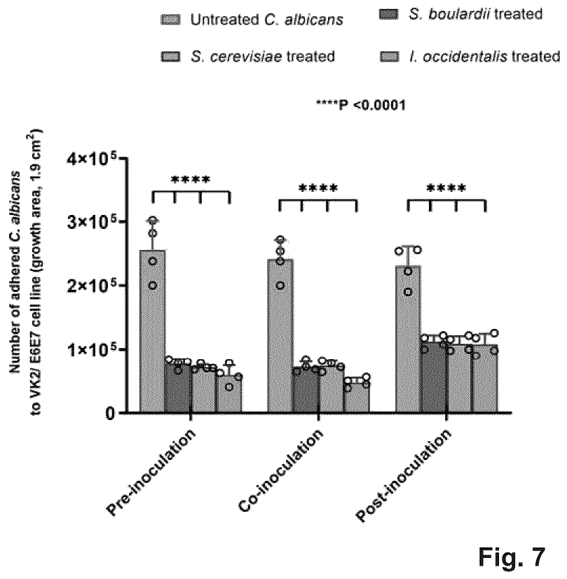

FIG. 7 shows a histogram demonstrating a significant improvement in that the probiotic yeasts prevent adhesion of C. albicans to the vaginal cell line VK2/E6E7;

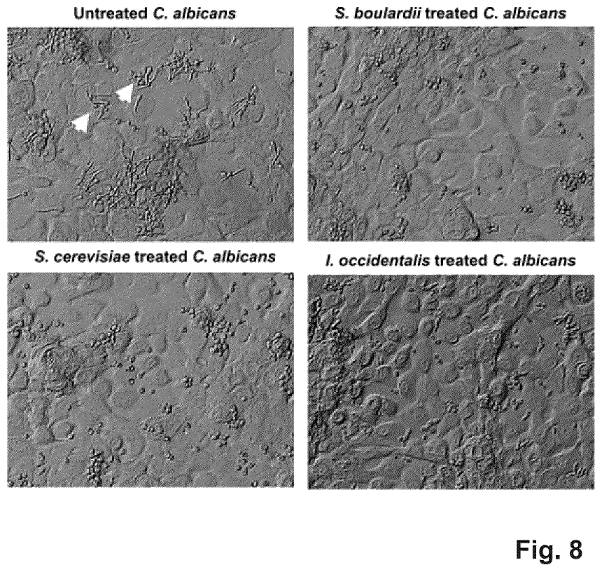

FIG. 8 shows micrographs demonstrating that the probiotic yeasts prevent adhesion of C. albicans to the vaginal cell line VK2/E6E7;

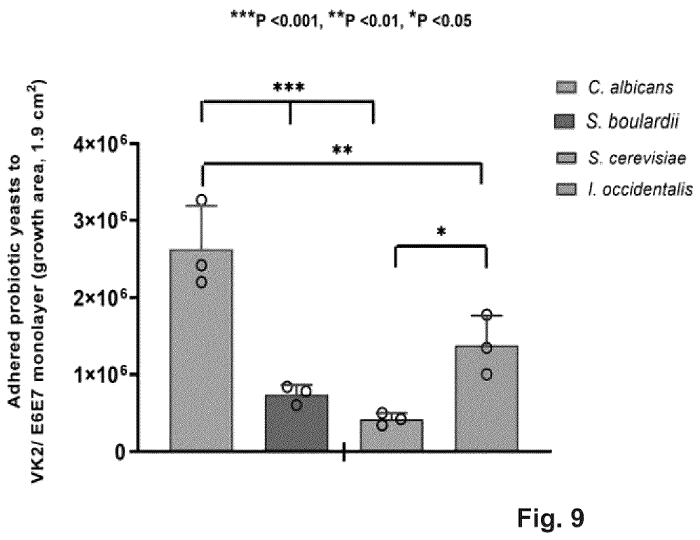

FIG. 9 shows a histogram indicating that probiotic yeasts attach to vaginal cells as compared to C. albicans, to the same cell lines; and

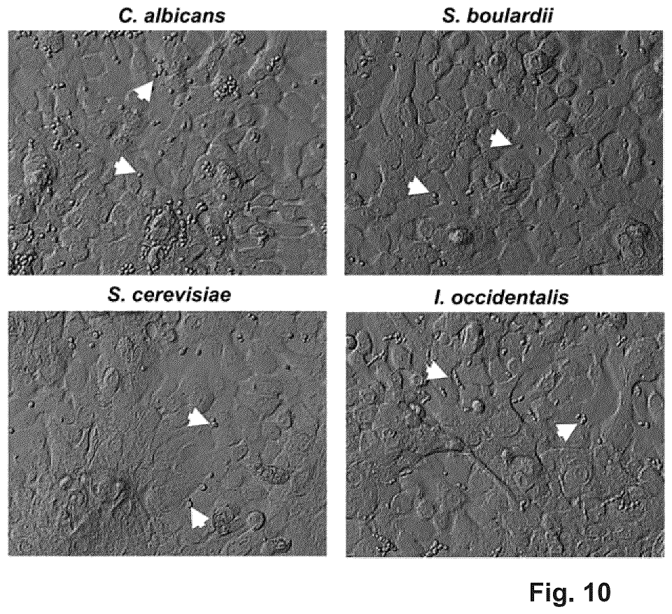

FIG. 10 is micrographs of adhered probiotic yeasts to vaginal epithelial cell lines.

### DETAILED DESCRIPTION

Long-term usage of antifungals contributes to the evolution of drug resistant Candida. Multidrug resistant C. albicans and non-albicans Candida strains are more commonly involved in vaginal candidiasis. These multidrug resistant super bugs (including Candida auris) have been designated as an urgent threat to public health by the Centers for Disease Control and Prevention as an (Centers for Disease control and prevention 2023). Configurations herein pursue treatment to defined beneficial microorganisms such as probiotics towards providing additional treatment option to vulvovaginal candidiasis. Hence, configurations herein present an alternative or combination therapy to treat drug resistant and/or recurrent vulvovaginal candidiasis and improve women health and wellness. Similar technology has been used successfully for gastrointestinal diseases, therefore intellectually should also be effective against vulvovaginitis.

Efficacy of probiotic yeasts has gained much attention in recent decades. The yeast Saccharomyces boulardii has an effect on several gastrointestinal and urinary tract diseases, and has been approved for use as a probiotic. Recently, two yeasts isolated from fermented beverages were shown to have probiotic effects against Candida albicans and other Candida strains. The first strain, Saccharomyces cerevisiae KTP, isolated from palm toddy, and the second strain, Pichia occidentalis ApC, isolated from fermented apple juice, were shown to control C. albicans filamentation and adhesion properties (Lohith, K. & Anu-Appaiah, K. A. Antagonistic effect of Saccharomyces cerevisiae KTP and Issatchenkia occidentalis). These strains were also shown to limit adhesion, filamentation, and biofilm formation of several non-albicans Candida species, including Candida tropicalis, Candida krusei, Candida glabrata, Candida parapsilosis, and Candida auris (Kunyeit, L., Kurrey, N. K., Anu-Appaiah, K. A. & Rao, R. P. Probiotic yeasts inhibit virulence of non-albicans Candida species). The strains further showed increased ahderence to the Caco-2 epithelial layer compared to S. cerevisiae. However, the genetic determinants of the beneficial phenotypes of these two strains are unknown.

Probiotic microbes are present in many fermented and cultured products across diverse cultures. Several have been isolated and marketed as probiotic supplements. To be classified as a probiotic, a microbe must exhibit beneficial effects and be properly identified by phenotypic and genomic methods (Reuter 2002). According to Qualified Presumption of Safety (QPS) developed by European Food Safety Authority (EFSA), definition of the taxonomy of a microorganism in feed and food application is a major safety parameter in the selection process. In other words, both the phenotype and genotype of a microbe must be defined before it can be called a probiotic. We have recently established a whole genome sequencing (WGS) pipeline called Prymetime. This tool can achieve higher genome contiguity and accuracy than previous approaches. Therefore, by applying Prymetime to probiotic yeast, WGS could improve taxonomic classification and provide insight into the genomic underpinnings of probiotic microbes. This could then be leveraged to genetically engineer targeted probiotic solutions for human health. Configurations herein apply Prymetime to two recently isolated yeast strains with beneficial properties.

Efficacy has been shown of two fermented food-derived (hence deemed safe) probiotic yeasts, Saccharomyces cerevisiae and Issatchenkia occidentalis on virulence of various Candida species in non-mammalian nematodes models of infection (Kunyeit, L., Kurrey, N. K., K A, A.-A. & Rao, R. P. Secondary metabolites from food-derived yeasts inhibit virulence of Candida albicans). These novel yeasts can prevent and reverse colonization of the gut and may be used as a probiotic or therapeutic against C. albicans as well as several Non-albicans Candida species including Candida auris. Subsequent translational studies in murine models will investigate the use of probiotic yeasts as an alternative and/or combination strategy to treat gut dysbiosis.

The linkage between the microbiome of the gut and mental health has been the source of much attention, generally labeled as the so-called “mind-body connection.” Disruption of the gut microbiome affects more than the gut. A common phenomenon known as “butterflies in your stomach” and resulting sensations emanating from your gut suggest that your brain and gut are connected. The gut and the nervous system have a complex bidirectional communication. For example, patients with Alzheimer's disease may harbor abnormal microbiome or manipulation of patient diet to treat depression and anxiety.

Probiotic yeasts uniquely combine human health benefits and tolerance phenotypes that enable survival in the gastrointestinal tract. They can produce beneficial metabolites and inhibit bacterial and fungal pathogens. They also can survive at human body temperature, withstand acidic and alkaline pHs similar to the digestive tract, and tolerate constituents of the digestive system like bile, gastric enzymes, and pancreatic enzymes. They also can adhere to gut epithelial cells. Perhaps unsurprisingly, probiotic yeasts are frequently found in fermented foods and beverages. Although several yeasts are known to have probiotic properties, only Saccharomyces boulardii has been commercialized and is prescribed to control and prevent gastrointestinal complications.

Among the physiologic ailments addressed by configurations herein, a fungal infection caused by one or more of Candida, Histoplasma, Blastomyces, Coccioides, Aspergillus, and Neurospora may be effectively treated. An ideal medium of the disclosed formulation may be as a capsule or tablet for treatment of gastrointestinal dysbiosis; and a suppository, a cream, a douche rinse, and a gel for treatment of vaginal or vulval dysbiosis. Alternatively, the subject may be an agricultural crop plant and the composition is formulated as a spray or a powder. Such crops or plants may include a banana, potato, tree fruit including peach, plum, mango, and olive, particularly when the fungus is at least one species selected from a Fusarium or a Phytophthora.

For human gut (gastrointestinal) ailments, configurations herein may be effective against gastrointestinal dysbiosis including symptoms of: colitis, Crohn's disease, Clostridium dificile infection, Alzheimer's disease, attention deficit disorder, depression, and diarrhea.

For human health concerns, the gut microbiome defines particular parameters that can eliminate certain species from proliferation. Environmental temperatures in a range of 25° C. to 42° C. need to be accommodated, as well as acidic and basic pH, such that tolerance to a low pH in a range of pH 2 to pH 4,and a high pH of pH 8 to a pH of 9. Genes so encoded for treatment via the gut need withstand these conditions.

Typical uses involve a warm-blooded animal, where a portion of the genes encode adherence to epithelial cells selected from at least one of gastrointestinal tract, epidermis, and vulva, and vagina; or the subject is a plant and a portion of the genes encodes for adherence to leaf epidermal cells. An amino acid alcohol, for example, tryptonol and phenylethanol, may be employed. An effective number of cells may include, for example, an effective dose is in a range of 10⁵ to 10⁹ cells for small mammal. Human usage may be more amendable to an effective dose in a range of 10⁶ to 10¹⁰ cells.

In still other configurations, a treatable epidermal dysbiosis is a tinea or dermatophytosis and the fungus is at least one genus selected from Epidermophyton, Microsporum, Malassezia and Trichophyton.

Using C. elegans, configurations herein investigate the neuronal connections between the gut and brain that allow the animal to avoid foods (infectious agents) that make it sick. Specific configurations investigate a role of the gustatory neurons (ASEL and ASER) that is concerned with taste in avoiding ingestion of a fungal pathogen C. albicans. Using two behavioral assays, the lawn occupancy assay and a modified version of the binary choice assay, configurations herein seek to establish that in the absence of the ASEL or ASER neuron C. elegans are unable to effectively detect that the fungal pathogen C. albicans is harming their gut post-infection. In wildtype C. elegans, C. albicans infection causes a distended gut and subsequent aversive behavior to the pathogen. In can be inferred that the ASE neurons mediate the aversive behavior. Using these behavioral experiments combined with molecular (life span assays) and genetic (mutant analysis) tools, configurations herein may probe the neuronal circuits between the gut and the brain. Since C. elegans faithfully recapitulates the anatomy, innate immunity and neuronal circuits of mammals, ultimately, this effort will yield a deeper understanding of how the gut microbiome regulates behavior and mental health.

Configurations herein demonstrate that these two food-derived probiotic yeasts, S. cerevisiae and I. occidentalis, prevent and reverse gut dysbiosis. Alternate configurations extend these beneficial results to a vaginal model using vaginal cell lines such as mouse models. In the disclosed configurations, a formulated composition for treating or preventing epithelial fungal infection and correcting dysbiosis in or on a subject, includes an effective dose of yeast cells containing a plurality of genes encoding functions of tolerance to a high temperature, tolerance to high and low pH, adherence to cells of the subject, and biosynthesis of amino acid alcohols. In general, the yeast cells are selected from at least one species selected from Saccharomyceacea and Issatchenkia families of yeasts.

In the case of Vulvovaginal Candidiasis, this ailment is currently treated with antifungal agents with limited efficacy. Furthermore, there are only three classes of antifungal agents available. The gut/vaginal microbiome plays an important role in keeping pathogens such as C. albicans at bay. Dysbiosis of the vaginal microbiome may be caused by hormonal flux, antibiotic therapy or a variety of contraceptives methods. Certain foods (cranberry products) tend to maintain the vagina microflora. Configurations herein demonstrate that fermented food-derived probiotic yeasts, Saccharomyces cerevisiae and Issatchenkia occidentalis are effective against vulvovaginal candidiasis. Such strategies that have been used for gastrointestinal ailments therefore should be effective in treating vulvovaginal candidiasis.

Whether used for targeting gut or vaginal health, the human physiology presents environmental conditions that the targeted therapy must adapt to. The genotypes underlying thermotolerance, pH, adherence, and metabolite biosynthesis of probiotic yeasts have been previously reviewed. Thermotolerance is essential because yeasts must survive at human body temperature. Genes with differential gene expression at 37° C. include the heat stress genes HSP26, SSA4, HSP82, HSP104, and GSY1, and the general stress genes TPS1 and GSY1. In addition, the SSQ1 gene in Pichia kudriavzevii plays a crucial role in long term heat stress response. Tolerance to pH—both acidic and alkaline—is essential to survive the gastrointestinal tract. A microarray analysis on acid shock responses using organic acids on S. cerevisiae revealed the stress response genes YGP1, TPS1, and HSP150 and the metal metabolism genes FIT2, ARN1, and ARN2 were induced under acidic shock stress. Another study showed a shorter adaptation time to acidic acid by the upregulation of genes AFT1 and HAA1. The genes FET4 CTR1 have shown increased expression in mild alkaline conditions. Adherence to intestinal epithelial cells is another important characteristic for probiotic yeast. The genes FLO1, FLO5, FLO9, and FLO10 are responsible for flocculation in S. cerevisiae, which is characterized by cell-cell adhesion. Along with flocculation, the related gene FLO11 has also shown association with floc formation, invasive growth, and substrate adhesion. Another possible gene involved with epithelial adhesion is ALA1, which has the ability to bind extracellular matrices. Beneficial metabolite biosynthesis includes acetate and propionate, which decrease luminal pH, induce bactericidal proteins, and increase short chain fatty acid production in the colon. Genes involved with the production of acetate and propionate include SIR2, HST1, HST2, HST3, and HST4. Further, aromatic alcohols tryptophol and phenylethanol were shown to inhibit filamentation of C. albicans. The genes ARO8 and ARO9 play a major role in producing these aromatic alcohols.

To more completely understand probiotic yeast genetics, whole genome sequencing and genetic tools are needed. For example, whole genome sequencing of Saccharomyces boulardii revealed that it was in fact a strain of S. cerevisiae, with a few notable differences in galactose metabolism and flocculation genes. Genetic tools for S. boulardii have also recently entered a new stage of refinement, opening up exciting possibilities for interrogating gentype-phenotype connections and creating designer probiotics. Thus, genome sequencing and genetic tools together make a firm foundation for engineering probiotic yeasts for human health. However, few genomes and genetic tools are available for probiotic yeast strains beyond S. boulardii. This prevents broader investigation into the generality, or uniqueness, of probiotic phenotypes. Here, we report characterization, genome sequencing, and genetic engineering of the probiotic yeast S. cerevisiae KTP. We show that KTP is from a different S. cerevisiae lineage than S. boulardii, but they both share mutations in flocculation genes, hinting at a general strategy for probiotic yeast adherence. Configurations herein depict the characterization and genome sequence of P. occidentalis ApC, placing it in the genus Issatchenkia, an underinvestigated branch of nonconventional yeasts with few probiotic strains recognized. This yeast appears to have a unique genotype producing probiotic phenotypes. The disclosed approach therefore advances the genomics of probiotic yeasts, particularly for gut and vaginal health.

Nanopore and Illumina reads were generated for the S. cerevisiae KTP and P. occidentalis ApC strains, and the reads were assembled into a genome using Prymetime v0.2. The assemblies were annotated using Augustus v3.2.3. The internal transcribed spacer (ITS) sequence for each strain's genome assembly was extracted based on the ITS1 and ITS4 primers and submitted to NCBI's BLASTN web server for species identification. The KTP strain is convincingly S. cerevisiae—the top 10 BLAST hits were all S. cerevisiae strains with percent identity above 99.5%. The species of the ApC strain was less clear—the top 10 BLAST hits were from several Pichia species. The ApC strain was previously identified as Pichia occidentalis (formerly Issatchenkia occidentalis), but we observed that other Pichia species returned higher BLAST scores than Pichia occidentalis.

FIG. 1 is a global phylogenetic position of the KTP strain in comparison to other S. cerevisiae strains. Referring to FIG. 1, the KTP strain was positioned in the global S. cerevisiae phylogenetic tree using data from the Saccharomyces cerevisiae 100 genomes project. The S. cerevisiae 100 genomes project used a set of 16 conserved regions—one from each chromosome—to construct a global phylogenetic tree. These conserved regions were extracted from the KTP genome assembly and added to the 100 genomes project data to construct a phylogenetic tree, shown in FIG. 1. The KTP strain is related to the Mosaic group, which has ancestry from two or more populations. The KTP strain was closest to the yjm1400, yjm1479, and yjm1401 strains, which had significant ancestry from Sake, North American, and Malaysian strains. Unlike many of the strains tested in the 100 yeast genomes project, these three strains did not have any Wine/European ancestry.

FIG. 2 shows a percent identity heatmap of BLASTp hits to known S. cerevisiae S288C probiotic-related proteins. Referring to FIG. 2, BLASTp hits from the S. cerevisiae KTP and S. boulardii unique28 strains with less than 80% query coverage are shown with black diagonal lines.

FIGS. 3A-3C show comparative genomics between S. cerevisiae S288c, S. boulardii unique28, and S. cerevisiae KTP for whole genome alignment with ProgressiveMauve. Referring to FIGS. 3A-3C, colored blocks indicate regions of high similarity. Red vertical lines designate a new contig in the genome assembly. Blocks below the center line are aligned sequences in the reverse direction.

FIG. 4 shows BUSCO genome completeness assessment of the three strains S. cerevisae S288c, S. boulardii unique28, and S. cerevisiae KTP. The white text shows the completeness score for each assembly.

FIG. 5 shows a Venn diagram of orthologous proteins shared or unique to the three strains: S. cerevisae S288c, S. boulardii unique28, and S. cerevisiae KTP.

FIG. 6 shows the number of BLASTp hits for the unique KTP proteins according to the top strain found.

As described above, configurations herein pursue whether fermented food-derived probiotic yeasts, Saccharomyces cerevisiae and Issatchenkia occidentalis are effective against vulvovaginal candidiasis. Such strategies have been used for gastrointestinal ailments therefore should be effective in treating vulvovaginal candidiasis.

A particular configurations tests whether food-derived yeasts, S. cerevisiae and I. occidentalis can prevent or reverse adhesion of C. albicans to vaginal epithelial cell-line, VK2/E6E7. Configurations herein pursue the prophylactic and therapeutic potential of fermented food-derived yeasts S. cerevisiae and I. occidentalis. Prophylactic potential will be tested by inoculating VK2/E6E7 cells with 10⁸ cells of the probiotics and then infected with 10⁶ cells of C. albicans. Therapeutic ability of food-derived yeasts will be tested by allowing C. albicans to colonize VK2/E6E7 monolayers, and then treating probiotic yeasts. In both conditions, unattached yeast cells will be removed and adhesion of C. albicans will be tested by serial dilution method followed by microscopy.

A further configuration evaluates the therapeutic potential of the food-derived probiotics, S. cerevisiae and I. occidentalis in a mammalian model of vulvovaginal candidiasis. Six to eight-week-old BALB/c female mice will be used in this study, each group contains eight mice. We will assess oral and vaginal administration of probiotic yeasts. We will test the prophylactic and therapeutic potential of fermented food-derived yeasts S. cerevisiae and I. occidentalis in both delivery modalities. A total of four experimental conditions are detailed below.

### Oral Administration

1. To test prophylactic potential, 10⁸ probiotic cells will be administered by oral gavage to mice for two-weeks prior to the Candida infection. In the third week mice will be infected with˜10⁵ cells of C. albicans by vaginal inoculation.
2. To test the therapeutic potential, probiotics will be used to female mice whose vagina is already colonized with C. albicans for a period of two weeks.

### Vaginal Administration

3. To test whether topical application of probiotic yeasts is effective, configurations herein apply S. cerevisiae, I. occidentalis to the vagina of the mouse prior to infection with C. albicans.
4. To test therapeutic potential, C. albicans infected mice will be treated with probiotic yeasts though intravaginal application for two weeks.

Development of vulvovaginal candidiasis has been previously described. S. boulardii will be used as reference probiotics and fluconazole (20 mg/Kg body weight) used as positive control in both the conditions. Effects of the probiotic yeasts on the virulence of C. albicans will be analyzed using transcriptomics, immunological (cytokine patterns), histopathological tools and microscopy.

A further configuration evaluates the safety profile of these fermented food-derived probiotic yeasts S. cerevisiae and I. occidentalis. Six to Eight-week-old gender balanced BALB/c mice (12 animal in each group, 6 male and 6 female) are employed in the safety studies of probiotic yeasts. 10e⁸ probiotic yeast cells will be administered orally for three weeks. Transcriptomics, histopathology and microscopy will be used to evaluate the safety profile of probiotics on mice. Result will be compared to a reference probiotic, S. boulardii and positive control, C. albicans, to evaluate a possible infection and pathogenesis with respect probiotics.

Multiple controls groups will be used to compare all results. Untreated, vehicle control, treatment with S. boulardii, a reference yeast that is commercially available probiotic and treatment with fluconazole, a front-line antifungal drug.

The disclosed configurations demonstrate efficacy of a probiotic yeast defined by the Saccharomyceacea and Issatchenkia families of yeasts. Effective mediums to address potency and delivery include method of screening a yeast isolate candidate for ability to remediate an epithelial dysbiosis disorder in a selected subject, including obtaining DNA from cells of the isolate, and characterizing presence and nucleic acid sequences of genes encoding functions of: tolerance to a high temperature, tolerance to high and low pH, adherence to epithelial cells of the selected subject in need of remediation of the disorder, and biosynthesis of amino acid alcohols. It should then be determined that the isolate candidate can remediate the dysbiosis disorders by containing the genes.

The disclosed method of treating, ameliorating or preventing a fungal infection in a subject using the disclosed compound includes providing a subject without an infection, or having a fungal infection in a tissue, and contacting infected tissue of the subject with an effective dose of yeast cells, in which the yeast cells are obtained from a strain having a genome that encodes a plurality of genes for functions of tolerance to a high temperature, tolerance to a high and a low pH, adherence to epithelial cells of the subject, and biosynthesis of amino acid alcohols.

FIG. 7 shows a histogram demonstrating a significant improvement in that the probiotic yeasts prevent adhesion of C. albicans to the vaginal cell line VK2/E6E7. In the trials depicted in FIG. 7, probiotic yeasts were treated to C. albicans in three different conditions and quantified by viable count method using Candida chrome agar. Incubation was 90 min. For the pre inoculation: probiotic yeasts, 10⁸/ml inoculated into vaginal epithelial monolayer for 30 min then C. albicans (10⁶/ml) infected and further incubated for 1 h. For Co inoculation: Probiotics and C. albicans co inoculated and incubated for 90 min. In post inoculation, First C. albicans infected for 30 min then treated with probiotic for 60 min. After incubation unadhered cells were removed by PBS washing. Adhered C. albicans were quantified by serial dilution method using Candida chrome agar.

FIG. 8 shows micrographs demonstrating that the probiotic yeasts prevent adhesion of C. albicans to the vaginal cell line VK2/E6E7. Referring to FIG. 8, the micrograph show respective probiotic treated and untreated C. albicans infected vaginal epithelial cell monolayer. Probiotic yeasts completely inhibited the filaments of C. albicans, indicated in white arrows in the micrograph, based on post-inoculation treatment.

Fig. 9 shows a histogram indicating that probiotic yeasts attach to vaginal cells as compared to C. albicans, to the same cell lines. This demonstrates many probiotic yeasts adhere to this cell line and compared to C. albicans (as a positive control, since Candida albicans is well known to attach biotic and abiotic surface). The quantification of adhered probiotic yeasts to vaginal epithelial cell line shows that 10⁸/ml of probiotics and/or C. albicans (positive control) inoculated and incubated for 90 min, then unadhered cells were removed and quantified by viable count method.

FIG. 10 is micrographs of adhered probiotic yeasts to vaginal epithelial cell lines. The individual micrographs for the respective samples demonstrate how a number of probiotic cells adhere to this cell line and compared to C. albicans as positive control, since Candida albicans is well known to attach biotic and abiotic surface.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A formulated composition for treating or preventing epithelial fungal infection and correcting dysbiosis in or on a subject, comprising an effective dose of yeast cells containing a plurality of genes encoding functions of tolerance to a high temperature, tolerance to high and low pH, adherence to cells of the subject, and biosynthesis of amino acid alcohols.

2. The composition according to claim 1, wherein the yeast cells are selected from at least one species selected from Saccharomyceacea and Issatchenkia families of yeasts.

3. The composition according to claim 1, wherein a first portion of the genes encode tolerance to environmental temperatures in a range of 25° C. to 42° C.

4. The composition according to claim 1, wherein a second portion of the genes encode tolerance to a low pH in a range of pH 2 to pH 4, and a high pH of pH 8 to a pH of 9.

5. The composition according to claim 1, wherein the subject is a warm-blooded animal and a third portion of the genes encode adherence to epithelial cells selected from at least one of gastrointestinal tract, epidermis, and vulva, and vagina; or the subject is a plant and the third portion of the genes encodes for adherence to leaf epidermal cells.

6. The composition according to claim 1, wherein a fourth portion of the genes encode enzymes for biosynthesis of at least one amino acid alcohol including tryptonol and phenylethanol.

7. The composition according to claim 1, further comprising at least one of tryptonol and phenylethanol.

8. The composition according to claim 5, wherein the subject is a rodent, a dog, a guinea pig, or a chicken, and the effective dose is in a range of 10⁵ to 10⁹ cells.

9. The composition according to claim 5, wherein the subject is a human, and the effective dose is in a range of 10⁶ to 10¹⁰ cells.

10. The composition according to claim 5, wherein the fungal infection is at least one selected from a species of Candida, Histoplasma, Blastomyces, Coccioides, Aspergillus, and Neurospora, and the composition is formulated as at least one selected from: a capsule or tablet for treatment of gastrointestinal dysbiosis; and a suppository, a cream, a douche rinse, and a gel for treatment of vaginal or vulval dysbiosis.

11. The composition according to claim 5, wherein the subject is an agricultural crop plant and the composition is formulated as a spray or a powder.

12. The composition according to claim 11, wherein the crop plant is banana, potato, tree fruit including peach, plum, mango, and olive, and the fungus is at least one species selected from: a Fusarium or a Phytophthora.

13. The composition according to claim 5, wherein the gastrointestinal dysbiosis is at least one selected from symptoms of: colitis, Crohn's disease, Clostridium dificile infection, Alzheimer's disease, attention deficit disorder, depression, and diarrhea.

14. The composition according to claim 5, wherein the epidermal dysbiosis is tinea or dermatophytosis and the fungus is at least one genus selected from Epidermophyton, Microsporum, Malassezia and Trichophyton.

15. A method of screening a yeast isolate candidate for ability to remediate an epithelial dysbiosis disorder in a selected subject, the method comprising:
obtaining DNA from cells of the isolate;
characterizing presence and nucleic acid sequences of genes encoding functions of: tolerance to a high temperature, tolerance to high and low pH, adherence to epithelial cells of the selected subject in need of remediation of the disorder, and biosynthesis of amino acid alcohols; and
determining that the isolate candidate can remediate the dysbiosis disorders by containing the genes.

16. A method of treating, ameliorating or preventing a fungal infection in a subject, the method comprising:
providing a subject without an infection, or having a fungal infection in a tissue;
contacting infected tissue of the subject with an effective dose of yeast cells, the yeast cells obtained from a strain having a genome that encodes a plurality of genes for functions of tolerance to a high temperature, tolerance to a high and a low pH, adherence to epithelial cells of the subject, and biosynthesis of amino acid alcohols; and
monitoring the infection in the tissue.

17. The method according to claim 16, wherein the subject has no infection, and the contacting step prevents appearance of a subsequent infection.

18. The method according to claim 16, wherein the subject has the infection and monitoring shows amelioration or cure of the infection.

19. The method according to claim 16, wherein the infection is gastrointestinal and following the contacting step the effective dose provides a probiotic microbiome that is unaffected by anti-bacterial therapy.

20. The method according to claim 16, wherein the fungal infection causative agent is Candida albicans or a Candida non-albicans yeast.

21. The method according to claim 20 wherein the agent is Candida auris and is resistant to anti-fungal drugs.
